(19) United States
(12) Patent Application Publication
(10) Pub. No.: US 2004/0234471 A1
(43) Pub. Date: Nov. 25, 2004

(54) Thickener for hair care products

(76) Inventors: Alberto Corbella, Como (IT); Christian Somigliana, Torno (IT)

Correspondence Address: COGNIS CORPORATION, PATENT DEPARTMENT, 300 BROOKSIDE AVENUE, AMBLER, PA, 19002, US

(21) Appl. No.: 10/480,677

(22) PCT Filed: Jun. 6, 2002
(86) PCT No.: PCT/EP02/06188

(30) Foreign Application Priority Data
Jun. 15, 2001 (DE) 101-29-034.9

(51) Int. Cl. A61K 7/06

(52) U.S. Cl. 424/70.1

(57) ABSTRACT

A cosmetic and/or pharmaceutical composition comprising a thickening concentrate, said concentrate containing: (a) an isostearic acid; (b) a fatty alcohol; and (c) an ethoxylated fatty alcohol.

### FIELD OF THE INVENTION

This invention relates generally to hair care and, more particularly, to thickener concentrates based on isostearic acid, fatty alcohols and ethoxylated fatty alcohols and to their use for the production of hair care preparations, primarily hair colorants.

### PRIOR ART

The thickeners used in the production of hair care preparations have to meet complex requirements. Whereas, on the one hand, they lead to rheological properties which provide for easy handling of the preparation, they are not expected to leave the hair feeling sticky after application or to reduce the volume of the hair by remaining on the hair fibers. International patent application WO 0018363 proposes numerous compounds as thickeners for shampoos and conditioners, including for example cellulose derivatives, starch, starch derivatives, natural polymers, sugars, electrolytes, block polymers or polyvinyl alcohol. However, not all the thickeners mentioned can be processed to storable, aesthetically pleasing formulations. International patent WO 9629980 describes hydroxyethyl cellulose as a thickener for a stable transparent dispersion in conditioning formulations. However, the incorporation of these thickeners is often temperature-dependent and time-consuming because cellulose ethers have to be preswollen in water. Accordingly, for simple processing, even in “cold” production processes, WO 9967017 discloses alkoxylated glucose derivatives with lipophilic substituents as thickeners.

Nevertheless, the care properties of the hair care products are not expected to be impaired by the use of thickeners. In the field of hair colorants, U.S. Pat. No. 4,357,141 claims formulations containing oxidation dyes and C₁₂₋₂₀ fatty acids as a base for establishing a cream-like consistency. These formulations have advantageous care properties.

Despite the numerous products on the market, the complex requirement profile for thickeners for use in hair care preparations has not yet been satisfactorily covered.

Accordingly, the problem addressed by the present invention was to provide a thickener for thickening hair care preparations which would simplify the process used for the production of these products. The thickener would provide for simple processing, would have high stability in storage and would satisfy the known requirements hair care products are expected to meet, including good hair care properties and high biological compatibility. The use of the thickener would improve the handling of the hair care preparations in practice without any adverse effect on hair conditioning performance, wet and dry combability, electrostatic charging, strength and breaking elongation.

### DESCRIPTION OF THE INVENTION

The present invention relates to thickener concentrates containing isostearic acid, fatty alcohols and ethoxylated fatty alcohols and to their use in hair care preparations, more particularly in hair colorants. The present invention also relates to hair care preparations which contain these thickener concentrates in the composition mentioned.

It has been found that thickener concentrates containing isostearic acid, fatty alcohols and ethoxylated fatty alcohols have excellent processing behavior. In contrast to conventional thickeners, they remain liquid under cold conditions (5° C.), are easy to incorporate, remain stable in storage over long periods and, if necessary, may be used as a starting product in the production of hair care preparations without the individual products having to be incorporated.

Besides simplified processing, the use of the thickener combination according to the invention leads to good dispersibility and hence to good color distribution in hair colorants. These advantages are largely attributable to the use of isostearic acid. In the production of shampoo hair coloring gels in particular, the use of the thickener combination according to the invention leads to a clear and easy-to-use liquid product whereas conventional thickeners containing oleic acid lead to cloudy and viscous products.

Fatty Alcohols

Fatty alcohols in the context of the invention are understood to be primary aliphatic alcohols corresponding to formula (I):

R¹OH (I)

where R¹ is an aliphatic, linear or branched hydrocarbon radical containing 6 to 22 carbon atoms and 0 and/or 1, 2 or 3 double bonds. Typical examples are caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof obtained, for example, in the high-pressure hydrogenation of technical methyl esters based on fats and oils or aldehydes from Roelen's oxo synthesis and as monomer fraction in the dimerization of unsaturated fatty alcohols. Preferred fatty alcohols are technical C₁₂₋₁₈ fatty alcohols such as, for example, coconut oil, palm oil, palm kernel oil and tallow fatty alcohol.

Ethoxylated Fatty Alcohols

Ethoxylated fatty alcohols are known from their production as fatty alcohol or oxoalcohol ethoxylates and preferably correspond to formula (II):

R²O(CH₂CH₂O)ₙH (II)

in which R² is a linear or branched alkyl and/or alkenyl group containing 6 to 22 carbon atoms and n is a number of 1 to 50. Typical examples are the adducts of, an average, 1 to 50, preferably 2 to 40 and more particularly 2 to 10 mol caproic alcohol, caprylic alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and the technical mixtures thereof obtained, for example, in the high-pressure hydrogenation of technical methyl esters based on fats and oils or aldehydes from Roelen's oxo synthesis and as monomer fraction in the dimerization of unsaturated fatty alcohols. Preferred ethoxylated fatty alcohols are products of the addition of 2 to 10 mol ethylene oxide onto technical C₁₂₋₁₈ fatty alcohols such as, for example, coconut oil, palm oil, palm kernel oil and tallow fatty alcohol.

Commercial Applications

Embodiments of the thickener concentrates according to the invention contain:

a) 4 to 15% by weight isostearic acid,

b) 1 to 5% by weight fatty alcohols and

c) 80 to 95% by weight ethoxylated fatty alcohols,

In a preferred embodiment, the thickener concentrates contain

a) 5 to 10% by weight isostearic acid,

b) 1 to 4% by weight fatty alcohols and

c) 85 to 93% by weight ethoxylated fatty alcohols,

Particularly preferred thickener concentrates contain:

a) 7 to 9% by weight isostearic acid,

b) 1 to 3% by weight fatty alcohols and

c) 88 to 92% by weight ethoxylated fatty alcohols.

The thickener concentrates are used in hair care preparations in quantities of 5 to 40% by weight, preferably in quantities of 10 to 35% by weight and more particularly in quantities of 20 to 30% by weight. They are mainly used in hair colorants and may be present in such formulations as solutions, foams, shampoos, creams, gels, lotions, conditioners or rinses.

Besides the thickener concentrates, the hair care preparations may contain dyes, surfactants, emulsifiers, superfatting agents, thickeners, polymers, silicone compounds, biogenic agents, film formers, preservatives and perfumes as further auxiliaries and additives.

Dyes

Keratin fibers, preferably in the form of human hair, are normally colored either with so-called substantive dyes or with oxidation colorants. Oxidation colorants are composed of a primary intermediate (oxidation base) and a secondary intermediate (shading component) and are not dyes in the true sense, but rather dye precursors. Oxidation bases are aromatic compounds which are nucleus-substituted by at least two electron-yielding groups (amino and/or hydroxy groups). For example, primary aromatic amines with another free or substituted hydroxy or amino group in the para or ortho position, diaminopyridine derivatives, heterocyclic hydrazones, 4-aminopyrazolone derivatives and 2,4,5,6-tetraaminopyrimidine and derivatives thereof are used. Special representatives are inter alia p-toluylenediamine, p-aminophenol, N,N-bis-(2-hydroxyethyl)-p-phenylene-diamine, 2-(2,5-diaminophenoxy)-ethanol, 1-phenyl-3-carboxyamido-4-amaino-5-pyrazolone and 4-amino-3-methylphenol, 2-(2-hydroxyethyl)-1,4-aminobenzene and 2,4,5,6-tetraaminopyrimidine.

Shading components are generally also aromatic compounds, but with groups readily oxidizable in the m-position of the ring, Components generally available include m-phenylenediamine derivatives, naphthols, resorcinol and resorcinol derivatives, pyrazolones, m-aminophenols and pyridine derivatives. Particularly suitable secondary intermediates are 1-naphthol, pyrogallol, 1,5-, 2,7- and 1,7-dihydroxynaphthalene, 5-amino-2-methylphenol, m-aminophenol, resorcinol, resorcinol monomethyl ether, m-phenylenediamine, 1-phenyl-3-methyl-5-pyrazolone, 2,4-dichloro-3-aminophenol, 1,3-bis-(2,4-diaminophenoxy)-propane, 2-chlororesorcinol, 2-chloro-6-methyl-3-aminophenol, 2-methylresorcinol, 2,5-dimethylresorcinol, 2,6-dihydroxypyridine and 2,6-diaminopyridine.

Suitable substantive dyes are, for example, dyes from the group of nitrophenylenediamines, nitroaminophenols, anthraquinones or indophenols such as, for example, the compounds known under the international names or commercial names of HC Yellow 2, HC Yellow 4, Basic Yellow 57, Disperse Orange 3, HC Red 3, HC Red BN, Basic Red 76, HC Blue 2, Disperse Blue 3, Basic Blue 99, HC Violet 1, Disperse Violet 1, Disperse Violet 4, Disperse Black 9, Basic Brown 16, Basic Brown 17, picramic acid and Rodol 9 R and also 4-amino-2-nitrodiphenylamine-2′-carboxylic acid, 6-nitro-1,2,3,4-tetrahydroquinoxalines, (N-2,3-dihydroxypropyl-2-nitro-4-trifluoromethyl)-aminobenzene and 4-N-ethyl-1,4-(2′-hydroxyethylamino)-2-nitrobenzene hydrochloride.

Besides synthetic dyes, naturally occurring dyes such as, for example, henna red, henna neutral, henna black, chamomile blossom, sandalwood, black tea, black alder bark, sage, logwood, madder root, catechu, sedre, alkanet, curcumin, hematoxylin and autin may also be used. The natural dyes cannot be unequivocally assigned to the two groups but, in the most common cases, fall into the group of substantive dyes.

Besides dye mixtures within the groups, mixtures of dyes from various groups may also be used. So far as other dye components are concerned, reference is specifically made to the Colipa List published by the Industrieverband Körperpflege und Waschmittel, Frankfurt. An overview of suitable dyes can also be found in the publication “Kosmetische Färbemittel” of the Farbstoffkommission der deutschen Forschungsgemeinschaft, Verlag Chemie, Weinheim, 1984, pp. 81-106.

Surfactants

Surfactants which may be present in the hair care preparations include nonionic, anionic, cationic and/or amphoteric or zwitterionic surfactants. Typical examples of anionic surfactants are soaps, alkyl benzenesulfonates, alkanesulfonates, olefin sulfonates, alkylether sulfonates, glycerol ether sulfonates, a-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds and esterquats, more particularly quaternized fatty acid trialkanolamine ester salts. Typical examples of amphoteric or zwitterionic surfactants are alkylbetaines, alkylamido-betaines, aminopropionates, aminoglycinates, imidazolinium betaines and sulfobetaines. The surfactants mentioned are all known compounds. Information on their structure and production can be found in relevant synoptic works, cf. for example J. Falbe (ed.), “Surfactants in Consumer Products”, Springer Verlag, Berlin, 1987, pages 54 to 124 or J. Falbe (ed.), “Katalysatoren, Tenside und Mineralöladditive (Catalysts, Surfactants and Mineral Oil Additives)”, Thieme Verlag, Stuttgart, 1978, pages 123-217. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, a-olefin sulfonates, ether carboxylic acids, alkyl oligoglucosides, fatty acid glucamides, alkylamidobetaines, amphoacetals and/or protein fatty acid condensates, preferably based on wheat proteins.

Emulsifiers

Suitable emulsifiers are, for example, nonionic surfactants from at least one of the following groups:

products of the addition of 2 to 30 mol ethylene oxide and/or 0 to 5 mol propylene oxide onto linear C₈₋₂₂ fatty alcohols, onto C₁₂₋₂₂ fatty acids, onto alkyl phenols containing 8 to 15 carbon atoms in the alkyl group and onto alkylamines containing 8 to 22 carbon atoms in the alkyl group;

alkyl and/or alkenyl oligoglycosides containing 8 to 22 carbon atoms in the alk(en)yl group and ethoxylated analogs thereof;

addition products of 1 to 15 mol ethylene oxide onto castor oil and/or hydrogenated castor oil;

addition products of 15 to 60 mol ethylene oxide onto castor oil and/or hydrogenated castor oil;

partial esters of glycerol and/or sorbitan with unsaturated, linear or saturated, branched fatty acids containing 12 to 22 carbon atoms and/or hydroxycarboxylic acids containing 3 to 18 carbon atoms and addition products thereof onto 1 to 30 mol ethylene oxide;

partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and addition products thereof onto 1 to 30 mol ethylene oxide;

mixed esters of pentaerythritol, fatty acids, citric acid and fatty alcohol according to DE 1165574 PS and/or mixed esters of fatty acids containing 6 to 22 carbon atoms, methyl glucose and polyols, preferably glycerol or polyglycerol,

mono-, di- and trialkyl phosphates and mono-, di- and/or tri-PEG-alkyl phosphates and salts thereof,

wool wax alcohols,

polysiloxane/polyalkyl/polyether copolymers and corresponding derivatives,

block copolymers, for example Polyethyleneglycol-30 Dipolyhydroxystearate;

polymer emulsifiers, for example Pemulen types (TR-1, TR-2) of Goodrich;

polyalkylene glycols and

glycerol carbonate.

The addition products of ethylene oxide and/or propylene oxide onto fatty alcohols, fatty acids, alkylphenols or onto castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layer enhancers for cosmetic formulations from DE 2024051 PS.

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide onto the partial glycerides mentioned are also suitable.

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxy-stearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylolpropane or pentaerythritol with lauric acid, cocofatty acid, tallow fatty acid, palmitic acid, stearic acid, oleic acid, behenic acid and the like optionally reacted with 1 to 30 mol ethylene oxide.

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Other Thickeners

Besides the thickener concentrate, the hair care preparations may contain other thickeners in reduced quantities such as, for example, polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates, polyvinyl alcohol and polyvinyl pyrrolidone or alkyl oligoglucosides and electrolytes, such as sodium chloride and ammonium chloride.

Polymers

Suitable polymers are, for example, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat polypeptides, polyethyleneimine, cationic silicone polymers such as, for example, amodimethicone, copolymers of adipic acid and dimethylaminohydroxypropyl diethylenetriamine (Cartaretine®, Sandoz), copolymers of acrylic acid with dimethyl diallyl ammonium chloride (Merquat® 550, Chemviron), polyaminopolyamides as described, for example, in FR 2252840 A and crosslinked water-soluble polymers thereof, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamido-propyl trimethylammonium chloride/acrylate copolymers, octylacryl-amide/methyl methacrylate/tert.-butylaminoethyl methacrylate/2-hydroxy-propyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones. Other suitable polymers and thickeners can be found in Cosmetics & Toiletries, Vol.108, May 1993, pages 95 et seq.

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates. A detailed overview of suitable volatile silicones can be found in Todd et al. in Cosm. Toil. 91, 27 (1976).

Biogenic Agents

In the context of the invention, biogenic agents are, for example, amino acids, protein hydrolyzates, ceramides, pseudoceramides, essential oils, plant extracts and vitamin complexes.

Film Formers

Other standard film formers are, for example, polyvinyl pyrrolidone, vinyl pyrrolidone/vinyl acetate copolymers, polymers of the acrylic acid series, quaternary cellulose derivatives, collagen, hyaluronic acid and salts thereof and similar compounds, copolymers of diallyl ammonium salts and acrylamides, quaternized vinyl pyrrolidone/vinyl imidazole polymers such as, for example, Luviquat® (BASF AG, Ludwigshafen/FRG), condensation products of polyglycols and amines, quaternized collagen polypeptides such as, for example, Lauryidimonium Hydroxypropyl Hydrolyzed Collagen (Lamequat® L, Grünau GmbH), polyethyleneimine, cationic silicone polymers such as, for example, amodimethicone or Dow Corning (Dow Corning Co., USA), copolymers of adipic acid and dimethylaminohy-droxypropyl diethylenetriamine (Cartaretine®, Sandoz/CH), polyaminopoly-amides as described, for example, in FR 2252840 A and crosslinked water-soluble polymers thereof, cationic guar gum such as, for example, Jaguar®CBS, Jaguar®C-17, Jaguar®C-16 of Celanese/USA, quaternized ammonium salt polymers such as, for example, Mirapol® A-15, Mirapol® AD-1, Mirapol® AZ-1 of Miranol/USA.

Preservatives

Suitable preservatives are, for example, phenoxyethanol, formaldehyde solution, parabens, pentanediol or sorbic acid and the other classes of compounds listed in Appendix 6, Parts A and B of the Kosmetikverordnung (“Cosmetics Directive”).

Perfume Oils and Perfumes

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxy-citronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

### EXAMPLES

TABLE 1
hair colorant

| Composition (INCI name) | Quantity (% by weight) |
|---|---|
| Thickener concentrate* | 30.0 |
| Isopropanol | 16.0 |
| Propylene glycol | 5.0 |
| Ammonia, 27% by weight | 4.0 |
| Monoethanolamine | 1.0 |
| Sodium sulfite | 0.5 |
| Oxidation dye | 1.5 |
| Sodium Laureth Sulfate | 1.0 |
| (Texapon ® NSO Cognis Düsseldorf) | |
| Water | to 100.0 |

*thickener concentrate consisting of 90% by weight C₁₂₋₁₄ fatty alcohol + 2EO (Dehydol ® LS2, Cognis, Düsseldorf), 7.5% by weight isostearic acid (Emersol ® 874, Cognis France) and 2.5% by weight coconut alcohol (Lorol ® C12-18, Cognis Düsseldorf)

The formulation was prepared by mixing at room temperature. The dye was predispersed with the sodium sulfite in part of the water.

TABLE 2
hair coloring gel

| Composition, base | Quantity (% by weight) |
|---|---|
| Thickener concentrate* | 28.7 |
| Isopropanol | 15.3 |
| Propylene glycol | 4.8 |
| Ammonia, 27% by weight | 4.0 |
| Monoethanolamine | 1.0 |
| Sodium Laureth Sulfate | 1.0 |
| (Texapon ® NSO, Cognis Düsseldorf) | |
| Water | to 100.0 |

*thickener concentrate consisting of 90% by weight C₁₂₋₁₄ fatty alcohol + 2EO (Dehydol ® LS2, Cognis, Düsseldorf), 8% by weight isostearic acid (Emersol ® 874, Cognis France) and 2% by weight coconut alcohol (Lorol ® C12-18, Cognis Düsseldorf)

The base (Table 2) was prepared by mixing at room temperature. 1 part of the base was then mixed with 1.25 parts of a 20% by volume aqueous hydrogen peroxide solution by slow stirring at room temperature to form a gel.

1-8. (cancelled).

9. A composition comprising a thickening concentrate, said concentrate containing:
(a) an isostearic acid;
(b) a fatty alcohol; and
(c) an ethoxylated fatty alcohol.

10. The composition of claim 9 wherein the isostearic acid is present in the concentrate in an amount of from about 4 to 15% by weight, based on the weight of the concentrate.

11. The composition of claim 9 wherein the isostearic acid is present in the concentrate in an amount of from about 5 to 10% by weight, based on the weight of the concentrate.

12. The composition of claim 9 wherein the isostearic acid is present in the concentrate in an amount of from about 7 to 9% by weight, based on the weight of the concentrate.

13. The composition of claim 9 wherein the fatty alcohol is present in the concentrate in an amount of from about 1 to 5% by weight, based on the weight of the concentrate.

14. The composition of claim 9 wherein the fatty alcohol is present in the concentrate in an amount of from about 1 to 4% by weight, based on the weight of the concentrate.

15. The composition of claim 9 wherein the fatty alcohol is present in the concentrate in an amount of from about 1 to 3% by weight, based on the weight of the concentrate.

16. The composition of claim 9 wherein the ethoxylated fatty alcohol is present in the concentrate in an amount of from about 80 to 95% by weight, based on the weight of the concentrate.

17. The composition of claim 9 wherein the ethoxylated fatty alcohol is present in the concentrate in an amount of from about 85 to 93% by weight, based on the weight of the concentrate.

18. The composition of claim 9 wherein the thickening concentrate is present in the composition in an amount of from about 5 to 40% by weight, based on the weight of the composition.

19. The composition of claim 9 wherein the thickening concentrate is present in the composition in an amount of from about 20 to 30% by weight, based on the weight of the composition.

20. A process for coloring hair comprising contacting the hair with a colorant composition containing a thickening concentrate, said concentrate containing:
(d) an isostearic acid;
(e) a fatty alcohol; and
(f) an ethoxylated fatty alcohol.

21. The process of claim 20 wherein the isostearic acid is present in the concentrate in an amount of from about 4 to 15% by weight, based on the weight of the concentrate.

22. The process of claim 20 wherein the isostearic acid is present in the concentrate in an amount of from about 7 to 9% by weight, based on the weight of the concentrate.

23. The process of claim 20 wherein the fatty alcohol is present in the concentrate in an amount of from about 1 to 5% by weight, based on the weight of the concentrate.

24. The process of claim 20 wherein the fatty alcohol is present in the concentrate in an amount of from about 1 to 3% by weight, based on the weight of the concentrate.

25. The process of claim 20 wherein the ethoxylated fatty alcohol is present in the concentrate in an amount of from about 80 to 95% by weight, based on the weight of the concentrate.

26. The process of claim 20 wherein the ethoxylated fatty alcohol is present in the concentrate in an amount of from about 88 to 92% by weight, based on the weight of the concentrate.

27. The process of claim 20 wherein the thickening concentrate is present in the composition in an amount of from about 5 to 40% by weight, based on the weight of the composition.

28. The process of claim 20 wherein the thickening concentrate is present in the composition in an amount of from about 20 to 30% by weight, based on the weight of the composition.
